(19) United States
(12) Patent Application Publication
(10) Pub. No.: US 2026/0341510 A1
(43) Pub. Date: Oct. 29, 2026

(54) ARTIFICIAL INTELLIGENCE BASED HEALTHCARE MANAGEMENT SYSTEMS AND METHODS

(71) Applicant: Johannes FARKAS, Wien (AT)

(72) Inventor: Johannes FARKAS, Wien (AT)

(21) Appl. No.: 19/487,371

(22) PCT Filed: May 24, 2023
(86) PCT No.: PCT/IB2023/055350

§ 371 Date: Nov. 24, 2025

(51) Int. Cl. G16H 50/20; G16B 20/20; G16B 40/00; G16H 10/20; G16H 10/60; G16H 20/10

(52) U.S. Cl. CPC G16H 50/20; G16B 20/20; G16B 40/00; G16H 10/20; G16H 10/60; G16H 20/10

(57) ABSTRACT

The present invention pertains broadly to the domain of artificial intelligence-based healthcare management systems and methods, particularly an AI system, method, and computer programme product dedicated to the maintenance, updating, and intelligent analysis of patient information for the purpose of delivering diagnostic and therapeutic insights.

### FIELD OF THE INVENTION

The present invention pertains broadly to the artificial intelligence-based healthcare management systems and methods, particularly an AI system, method, and computer programme product dedicated to the maintenance, updating, and intelligent analysis of patient information for the purpose of delivering diagnostic and therapeutic insights.

### BACKGROUND OF THE INVENTION

In the medical field, many different tools are available for learning about and treating patient conditions. Traditionally, physicians would physically examine patients and draw upon a vast array of personal knowledge gleaned from years of study to identify problems and conditions experienced by patients and determine appropriate treatments. Over the past decades, and particularly in recent years, a wide array of further reference materials have become available to the practitioner that greatly expand the resources available and enhance and improve patient care.

Doctors keep long-term medical records for all patients. These medical records are usually stored in the records area of the practice. Unfortunately, when a patient changes doctors, there is no easy way to ensure that the new doctor has access to the patient's previous medical records without copying the patient's medical records and sending a copy to the next doctor.

Electronic medical records are designed to help doctors monitor their patients. Unfortunately, communication, the exchange of information, and access to medical information between medical professionals are often not possible because of a lack of standardization.

Traditionally, vast amounts of patient medical information are stored across the globe in specialized formats within medical offices, workplaces, hospitals, government agencies, and other institutions. This occurs even when these locations are inconvenient for the patients or when the patients are too ill to access them. While this medical information can be valuable for patient management, there is currently no reliable method to swiftly disseminate this information to organizations that could assist the patient. These organizations might include doctors, hospitals, public and social service agencies, offices, and third-party payers. This lack of efficient information distribution is particularly problematic in urgent situations where timely treatment is required.

Efforts to create medical records based on common medical records and electronic medical records have facilitated access to medical information. But the real problem remains that doctors, hospitals, insurance companies, or patients do not get timely information when they need it. Most medical records are now stored in doctor's offices, hospitals, or archives.

Another challenge posed by electronic medical records is their tendency to be repetitive and incomplete compared to traditional medical records. The organisation and management of data storage are laborious, with the majority of data processing conducted for a specific purpose rather than for comprehensive patient data management, creating a frustratingly inefficient system. For instance, a hospital might maintain multiple distinct files for patient medical records: one for financial transactions and billing, another for documenting patient symptoms, and yet another for recording the results of blood tests.

In many cases, doctors currently do not have access to patients' past medical records, making it difficult to treat patients referred by counsellors. The patient's medical records are usually in the primary care centre and are therefore currently inaccessible to the referring physician. Tests and diagnostics can take place without access to the patient's vital medical records for a variety of reasons, such as offices closing or charts not being available on time. It is often possible to repeat tests, especially in the context of emergency treatment, where the lack of this information would be crucial.

The inventor of the current application has delineated all publicly disclosed aspects of a patient's health history and proposed computer-adaptable systems, processes, and resources for providing patients with portable, secure, and up-to-date digital medical records that are accessible online. The present invention is pertinent to urgent situations in patient care as well as instances where the patient is relocating or transitioning to a new healthcare provider. Moreover, it offers a solution to standardize the storage and access of medical records, thereby enhancing cooperation and communication among different healthcare providers and institutions.

### SUMMARY OF THE INVENTION

The present invention relates to an AI system, method, and computer programme product for patient diagnosis and treatment recommendations, guided by advanced AI algorithms for diagnosing diseases and recommending treatments by analyzing all available data .. The system is crafted to bolster the precision and efficiency of healthcare delivery, equipping healthcare professionals with the most current information and treatment alternatives drawn from the latest research findings and clinical data.

In an exemplary embodiment of the present invention, an AI system, method, and computer programme product for providing patients with access to or control over their medical records are disclosed. A flexible, portable, patient-controlled system for storing, maintaining, and processing medical information may be provided. The main focus of the invention is to assist doctors in diagnosing diseases and recommending treatments by analyzing all available data.

The present invention offers a comprehensive, interconnected AI system(s) and method(s) leveraging artificial intelligence to facilitate patient diagnosis and treatment recommendations. By connecting multiple portals and data sources, healthcare professionals gain access to a wealth of information, enabling well-informed decisions based on the latest research and clinical data.

In an embodiment of the invention, the main AI system, method, and computer programme product are providing data to patients, doctors, and other agencies by gathering healthcare data from other attached AI-based systems.

In a representative embodiment, the main AI system could comprise: a minimum of one medical information source (AI systems or AI enabled sites); at least one personal medical data source pertaining to one or more patients; and one or more servers. The medical information and personal medical data would be accessible to these servers. Each server would incorporate an artificial intelligence (AI) component tasked with analyzing the personal medical data in conjunction with the medical information and identifying any issues necessitating further attention by the patient or an authorized external entity. The system would also include a real-time communication link enabling bi-directional interaction with an authorized external entity. Importantly, the personal medical data of a patient would be accessible to that individual.

In various embodiments, the main AI system gathers medical history and other patient information in real time, connects to AI systems or AI enabled sites for comprehensive electronic health records, obtains diagnostic imaging data, and fetches molecular biology data. Data regarding patients' medications, effectiveness, side effects, and drug interactions must also be provided to the main system.

In an embodiment of the present invention, there is a main AI system for getting various variables of medical history and other information about a patient in real time from other AI systems or AI-enabled sites.

In several embodiments, AI systems or AI-enabled sites are provided for gathering medical history and patient information, obtaining comprehensive electronic health records, diagnostic imaging data, and molecular biology data. Data regarding patients' medications, effectiveness, side effects, and drug interactions must also be provided by these methods.

In another embodiment of the invention, there is provided a main AI system that is connected to a plurality of AI systems or AI enabled sites that provides comprehensive data related to patients' electronic health records, which includes their medical history, diagnoses, treatments, and medications.

In an embodiment of the invention, AI systems or an AI-enabled site are provided for obtaining genomic analysis to the main AI system.

Laboratory analysis may also be considered by the main AI system by obtaining the same from other suitable AI systems or AI enabled sites.

The electronic health records of a patient are also being considered, analyzed and provided to the main AI system through other independent, dedicated AI systems or AI enabled sites.

In another embodiment of the invention, an AI system(s) or AI enabled site(s) is/are provided for obtaining diagnostic imaging data, including X-rays, MRIs, CT scans, and other radiological images of patients, from a data provider site to the main AI system.

Pathological data is also being considered and analyzed by independent AI systems or AI enabled sites, and this interpreted data is provided to the main AI system.

In an embodiment of the invention, there is a main AI system for fetching data from other AI systems or AI enabled sites for molecular biology data, including proteomics and metabolomics of a patient.

Data about patients' medications, their effectiveness, side effects, and any interactions between drugs is also required to be provided to the main AI system through independent AI systems or AI enabled sites.

An AI system or AI enabled site is also provided therein that will do the anamnesis with the patient, asking the right questions based on the patient's answers, supporting the physician, and taking out human bias, and provide these results to the main AI system.

In an embodiment of the invention, the system comprises a plurality of portals (AI systems or AI enabled sites), each designed to connect different players in the healthcare system. These portals include PatientConnect. ai, which enables patients to connect with healthcare professionals and access health information and resources; GenomicAnalysis. ai, providing genomic analysis to healthcare professionals; LabResults. ai, offering laboratory analysis to healthcare professionals; PatientRecords. ai, allowing healthcare professionals to access electronic health records and patient data; DiagnosticImaging.ai, delivering imaging analysis to healthcare professionals; DiagnosticPathology.ai, supplying pathology analysis to healthcare professionals; MolecularBiology.ai, furnishing molecular biology analysis to healthcare professionals; and Pharmacotherapy.ai, presenting AI-guided pharmacotherapy recommendations along with Anamnesis.ai is an AI-based system that will do the anamnesis with the patient, asking the right questions based on the patient's answers, supporting the physician, and taking out human bias.

In an embodiment of the invention, the data collected from the above AI systems or AI enabled sites is interpreted by the main AI system and provides diagnostic and treatment suggestions to healthcare professionals and other users.

In an embodiment of the invention, there is provided a method wherein the main AI system gathers medical history and other patient information in real time and connected to AI systems or AI enabled sites providing comprehensive electronic health records, obtains diagnostic imaging data, and fetches molecular biology data. Data regarding patients' medications, effectiveness, side effects, and drug interactions must also be provided to the main system.

In an embodiment of the invention, there is provided a method wherein the main AI system gets various variables of medical history and other information about a patient in real time from other AI systems or AI-enabled sites.

In an embodiment of the invention, there is provided a method wherein AI systems or AI-enabled sites are provided for gathering medical history and patient information, obtaining comprehensive electronic health records, diagnostic imaging data, and molecular biology data, Data regarding patients' medications, effectiveness, side effects, and drug interactions must also be provided by these methods. The collected data is then provided to the main AI system.

In another embodiment of the invention, there is provided a method by which a main AI system that is connected to a plurality of AI systems or AI-enabled sites provides comprehensive data related to patients' electronic health records, which includes their medical history, diagnoses, treatments, and medications.

In an embodiment of the invention, a method is provided for obtaining genomic analysis from other AI systems or AI-enabled sites to the main AI system.

In an embodiment of the invention, there is provided a method wherein laboratory analysis may also be considered by the main AI system by obtaining the same from other suitable AI systems or AI enabled sites.

In an embodiment of the invention, a method is provided wherein the electronic health records of a patient are also considered, analyzed and provided to the main AI system through other independent dedicated AI systems or AI enabled sites.

In another embodiment of the invention, a method is provided wherein, an AI systems or AI-enabled sites is provided for obtaining diagnostic imaging data, including X-rays, MRIs, CT scans, and other radiological images of patients, for the main AI system.

In an embodiment of the invention, there is provided a method wherein, pathological data is also considered and analyzed by independent AI systems or AI enabled site and these interpreted data are provided to the main AI system.

In an embodiment of the invention, there is provided a method wherein the main AI system fetches data from other AI systems or AI-enabled sites for molecular biology data, including proteomics and metabolomics, of a patient.

In another embodiment of the invention, a method is provided wherein data about patients' medications, their effectiveness, side effects, and any interactions between drugs is also required to be provided to the main AI system through independent AI systems or AI-enabled sites.

In another embodiment of the invention, there is provided a method wherein an AI system or AI enabled site is also provided that will do the anamnesis with the patient, asking the right questions based on the patient's answers, supporting the physician, and taking out human bias, and provide results to the main AI system.

In an embodiment of the invention, there is provided a method wherein, the system used, comprises a plurality of portals (AI systems or AI enabled sites), each designed to connect different players in the healthcare system. These portals include PatientConnect. ai, which enables patients to connect with healthcare professionals and access health information and resources; GenomicAnalysis.ai, providing genomic analysis to healthcare professionals; LabResults.ai, offering laboratory analysis to healthcare professionals; PatientRecords.ai, allowing healthcare professionals to access electronic health records and patient data; DiagnosticImaging.ai, delivering imaging analysis to healthcare professionals; DiagnosticPathology.ai, supplying pathology analysis to healthcare professionals; MolecularBiology.ai, furnishing molecular biology analysis to healthcare professionals; and Pharmacotherapy.ai, presenting AI-guided pharmacotherapy recommendations along with Anamnesis.ai is an AI-based system that will do the anamnesis with the patient, asking the right questions based on the patient's answers, supporting the physician, and taking out human bias.

In another embodiment of the invention, there is provided a method wherein the data collected from above AI systems or AI enabled sites are interpreted by the main AI-based system and provide diagnostic and treatment suggestions to healthcare professionals and other users.

### BRIEF DESCRIPTION OF THE DRAWINGS

The foregoing and other features and advantages of the invention will be apparent from the following, more particular description of exemplary embodiments of the invention, as illustrated in the accompanying drawings.

FIG. 1 depicts a block diagram of an exemplary embodiment of a system according to the present invention

FIG. 2 illustrates an exemplary computing device as may be used as a PMI (patient medical information) device, according to an exemplary embodiment of the present invention;

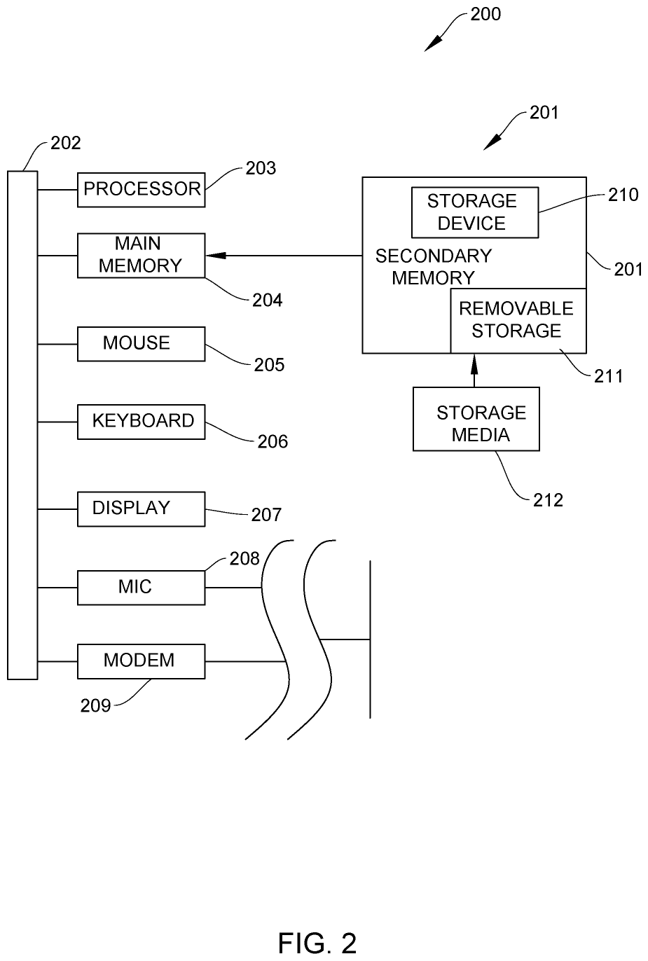

FIG. 3 illustrates an exemplary flow chart of user interaction with an exemplary system according to the present invention;

### DETAILED DESCRIPTION OF THE INVENTION

The present invention pertains to a system, method, and computer programme product for providing patients with access to or control over their medical records. A flexible, portable, patient-controlled system for storing, maintaining, and processing medical information may be provided. The main focus of the invention is to assist doctors in diagnosing diseases and recommending treatments by analyzing all available data.

In this context, “software” refers to codes or instructions that enable a computer or a part of it to function. Examples of software components include code fragments, informational codes, pre-compiled code, compiled code, translated code, computer programmes, and logical algorithms.

A “computer” may refer to one or more apparatus or one or more systems that are capable of processing input, processing the structured input according to certain rules, and producing the results of the processing as output. A typical computer may include: a laptop, desktop, handheld device, tablets, etc.; a computer having a single or multiple processors, or multi-core processors, which may operate in parallel or not in parallel; a computer; a laptop; a server; a client; a smart television; a telecommunications device with internet access; and an apparatus that may accept and process data in accordance with one or more stored software programmes, may generate results, and typically may include input, output, storage, arithmetic, logic, and control units.

The present invention relates to an AI system, method, and computer programme product for patient diagnosis and treatment recommendations, guided by advanced AI algorithms. The system is crafted to bolster the precision and efficiency of healthcare delivery, equipping healthcare professionals with the most current information and treatment alternatives drawn from the latest research findings and clinical data.

In an exemplary embodiment of the present invention, an AI system, method, and computer programme product for providing patients with access to or control over their medical records are disclosed. A flexible, portable, patient-controlled system for storing, maintaining, and processing medical information may be provided. The main focus of the invention is to assist doctors and patients in diagnosing diseases and recommending treatments by analyzing all available data.

The present invention offers a comprehensive, interconnected AI system(s) and method(s) leveraging artificial intelligence to facilitate patient diagnosis and treatment recommendations. By connecting multiple portals and data sources, healthcare professionals gain access to a wealth of information, enabling well-informed decisions based on the latest research and clinical data.

In an embodiment of the invention, the main AI system, method, and computer programme product is providing data to patients, doctors and other agencies by gathering healthcare data from other attached AI based systems.

In a representative embodiment, the main AI system could comprise: a minimum of one medical information source (AI systems or AI enabled sites); at least one personal medical data source pertaining to one or more patients; and one or more servers. The medical information and personal medical data would be accessible to these servers. Each server would incorporate an artificial intelligence (AI) component tasked with analyzing the personal medical data in conjunction with the medical information and identifying any issues necessitating further attention by the patient or an authorized external entity. The system would also include a real-time communication link enabling bi-directional interaction with an authorized external entity. Importantly, the personal medical data of a patient would be accessible to that individual.

In various embodiments, the main AI system and method gather medical history and other patient information in real time, connect to AI systems or AI enabled sites providing comprehensive electronic health records, obtain diagnostic imaging data, and fetch molecular biology data. Data regarding patients' medications, effectiveness, side effects, and drug interactions must also be provided to the main system.

In an embodiment of the present invention, there is a main AI system and method for getting various variables of medical history and other information about a patient in real time from other AI systems or AI enabled sites.

In several embodiments, AI systems and methods are provided for gathering medical history and patient information, obtaining comprehensive electronic health records, diagnostic imaging data, and molecular biology data. Data regarding patients' medications, effectiveness, side effects, and drug interactions must also be provided by these methods.

In another embodiment of the invention, there is provided a main AI system that is connected to a plurality of AI systems or AI enabled sites that provides comprehensive data related to patients' electronic health records, which includes their medical history, diagnoses, treatments, and medications, and a method thereof.

In an embodiment of the invention, AI systems or AI-enabled sites and methods are provided for obtaining genomic analysis to the main AI system.

Laboratory analysis may also be considered by the main AI system by obtaining the same from other suitable AI systems or AI enabled sites and methods thereof.

The electronic health records of a patient are also being considered, analyzed and provided to the main AI system through other independent AI systems or AI-enabled sites by a method.

In another embodiment of the invention, an AI-enabled system and method are provided for obtaining diagnostic imaging data, including X-rays, MRIs, CT scans, and other radiological images of patients, from an AI system or AI-enabled sites to the main AI system.

Pathological data is also being considered and analyzed by independent AI systems or AI enabled sites, and this interpreted data is provided to the main AI system and a method thereof.

In an embodiment of the invention, there is a main AI system and method therein for fetching data from other AI systems or AI enabled sites for molecular biology data, including proteomics and metabolomics, of a patient. Data about patients' medications, their effectiveness, side effects, and any interactions between drugs is also required to be provided to the main AI system through independent AI systems or AI-enabled sites by a method.

AI systems or AI enabled sites and method thereof are also provided therein that will do the anamnesis with the patient, asking the right questions based on the patient's answers, supporting the physician, taking out human bias, and provide these results to the main AI system.

In particular, the invention includes a system and method with a plurality of portals connecting different players in the healthcare system (AI systems or AI enabled sites). These portals consist of:
PatientConnect.ai enables patients to connect with healthcare professionals and access health information and resources.
GenomicAnalysis.ai provides genomic analysis to healthcare professionals.
LabResults.ai offers laboratory analysis to healthcare professionals.
PatientRecords.ai allows healthcare professionals to access electronic health records and patient data.
DiagnosticImaging.ai delivers imaging analysis to healthcare professionals.
DiagnosticPathology.ai supplies pathology analysis to healthcare professionals.
MolecularBiology.ai provides molecular biology analysis to healthcare professionals.
Pharmacotherapy.ai presents AI-guided pharmacotherapy recommendations to healthcare professionals.
Anamnesis.ai is an AI system or AI enabled site that will do the anamnesis with the patient, asking the right questions based on the patient's answers, supporting the physician, and taking out human bias.

The system may store and analyse medical information on a personal medical information device (which includes wearables) that may be carried by a patient. Other data, such as patients' electronic health records, which include their medical history, diagnoses, treatments, and medications, diagnostic imaging data, including X-rays, MRIs, CT scans, and other radiological images of patients' molecular biology data, including proteomics, metabolomics of a patient, medications, their effectiveness, side effects, and any interactions between drugs, is also being provided to the main artificial intelligence (AI) system.

These data are collected from various sources (AI systems or AI enabled sites), such as PatientConnect.ai, which enables patients to connect with healthcare professionals and access health information and resources; and smartwear.ai, which provides real-time health tracking and analysis through wearable devices. GenomicAnalysis.ai, providing genomic analysis to healthcare professionals; LabResults.ai, offering laboratory analysis to healthcare professionals; PatientRecords.ai, allowing healthcare professionals to access electronic health records and patient data; DiagnosticImaging.ai, delivering imaging analysis to healthcare professionals; DiagnosticPathology.ai, supplying pathology analysis to healthcare professionals; MolecularBiology.ai, furnishing molecular biology analysis to healthcare professionals; and Pharmacotherapy.ai, presenting AI-guided pharmacotherapy recommendations to healthcare professionals. However, other data may also be collected from other government or non-government sources to make the diagnostic and treatment recommendations. The output is not limited to treatment alone; it may also provide other recommendations to assist doctors in diagnosing diseases and recommending treatments by analyzing all available data.

The system further comprises an artificial intelligence (AI)-guided patient diagnosis and treatment recommendation system and method using a combination of cloud-based servers, edge computing devices, and a distributed data storage infrastructure. The cloud servers handle the majority of the data processing and AI model training, while edge computing devices (e.g., smartphones, tablets, or specialized medical devices) allow for real-time data processing and analysis closer to the source of data generation. The distributed data storage infrastructure ensures secure and efficient access to medical data. The system uses both wired and wireless networks to facilitate communication between different components and users.

The main artificial system may call as innovativetechnologies.ai is main AI engine receiving, processing and analyzing data from PatientConnect.ai, which enables patients to connect with healthcare professionals and access health information and resources; GenomicAnalysis.ai, providing genomic analysis to healthcare professionals; LabResults.ai, offering laboratory analysis to healthcare professionals; PatientRecords.ai, allowing healthcare professionals to access electronic health records and patient data; DiagnosticImaging.ai, delivering imaging analysis to healthcare professionals; DiagnosticPathology.ai, supplying pathology analysis to healthcare professionals; MolecularBiology.ai, furnishing molecular biology analysis to healthcare professionals; and Pharmacotherapy.ai, presenting AI-guided pharmacotherapy recommendations to healthcare professionals. However, other data may also be collected from other government or non-government sources to make the diagnostic and treatment recommendations. The output is not limited to treatment alone; it may also provide other recommendations to assist doctors in diagnosing diseases and recommending treatments by analyzing all available data. The AI component should not only identify issues requiring follow-up but also provide comprehensive treatment recommendations based on evidence levels found in TreatmentPlan.ai, GoldStandard.ai, and StateOfTheArt.ai.

Embodiments of the present invention may allow the updating of a patient's medical record and provide clinical suggestions based on any updated patient medical information. An embodiment may allow the patient, hospital, physician, insurance providers, and any related health care domain to access any pertinent information related to patient care in an online manner.

Embodiments of the present invention may allow a patient to monitor his own medical report. This will help the patients do the following: Patients may compare their care to the accepted standard medical record guidelines. Patients may compare their medical condition with any disease process or population similar to their medical condition or any other comparison regarding any medical situation based on data, diagnosis, and treatment protocols in an exemplary system. Patients can assess how well a particular doctor, hospital, or other related health domain is caring for them in relation to the standard of care or accepted practices. Patients may interact with any pertinent health care domains through an exemplary system to provide information regarding their medical situation.

Embodiments of the present invention may alert the patient, his primary care physician, and any health-related monitoring agency that monitors health-related demographics that he or she may not be on appropriate medications to treat his or her medical conditions.

### DETAILED DESCRIPTION OF THE DRAWINGS

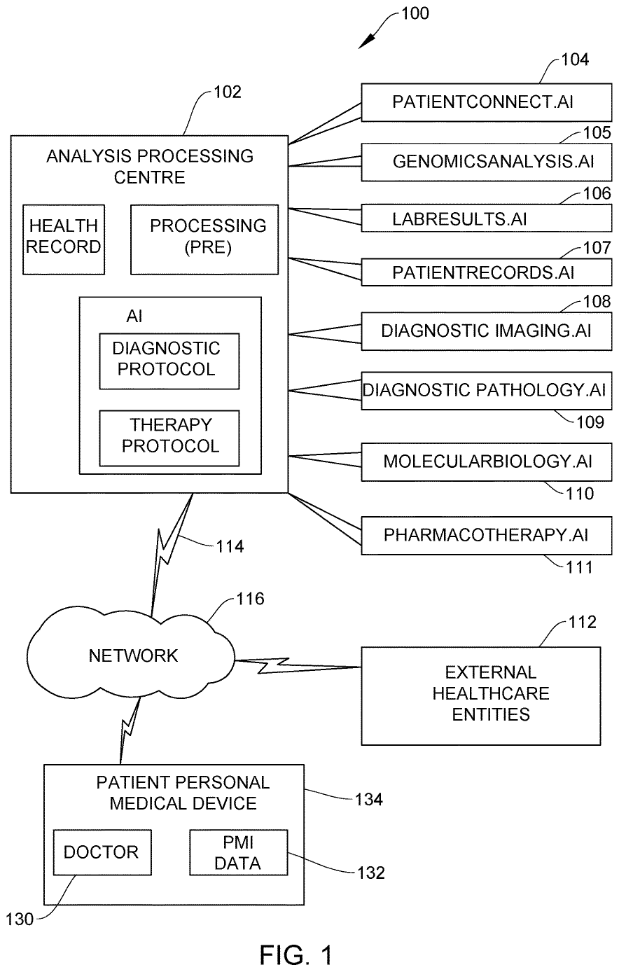

As seen in the block diagram (100) in FIG. 1, the present invention may include a centralized component, referred to hereinafter as the analysis processing centre (102) or main AI system, where health records, medical information, and patient data may be scanned, analyzed, or stored, and where the main artificial intelligence processes reside and take place. The analysis processing centre (102) or main AI system may be centralized, distributed, or functionally centralized. The analysis processing centre (102) or main AI system may include and operate on one or more server computers. The analysis processing centre (102) or main AI system gathers data from one or more databases or websites (AI systems or AI enabled sites) (104, 105, 106, 107, 108, 109, 110, 111 etc.) and may be furthermore. The databases and websites may be resident on the servers or may be separate from and accessible from the servers. The analysis processing centre (102) or main AI system may include a variety of network communication ports (114) for communicating with external healthcare entities (112) through a network or cloud (116).

Analysis processing centre (102) or main AI system may include a processing (pre) unit 130, and the same may be software or hardware that organizes, maintains, edits, and processes the incoming patient data and medical information from databases or websites (AI systems or AI enabled sites) (104, 105, 106, 107, 108, 109, 110, 111 etc.).

Analysis processing centre (102) or main AI system may include various artificial intelligence components, such as AI component and AI derivation component 124, comprising diagnostic protocol (126) and therapy protocol (128).

The analysis processing centre (102) or main AI system is in communication with a patient's personal medical device (PMD) (134). The device may store the patient's medical records,

PMI (patient medical information) database (132) for saving and storing patient's information If the patient also has a PMD (134), the patient's medical data may also be stored or accessible on the medical device.

The computer system (200) may include one or more processors (203) that may be connected to a communications bus, cross-over bar, network, etc. (204). The computer system (200) may include a display interface (202) that may forward to a communications bus, cross-over bar, network, etc. (204).

The computer system (200) may also include, but may not be limited to, a main memory (201), random access memory (RAM), and a secondary memory. The secondary memory (210) may include, for example, a hard disc drive (211) or a removable storage drive (212). A computer (200) may also include an input device such as, e.g., a mouse or other pointing device such as a digitizer, a keyboard, or other devices.

Computer systems (200) also include output devices, such as, e.g., displays and display interfaces.

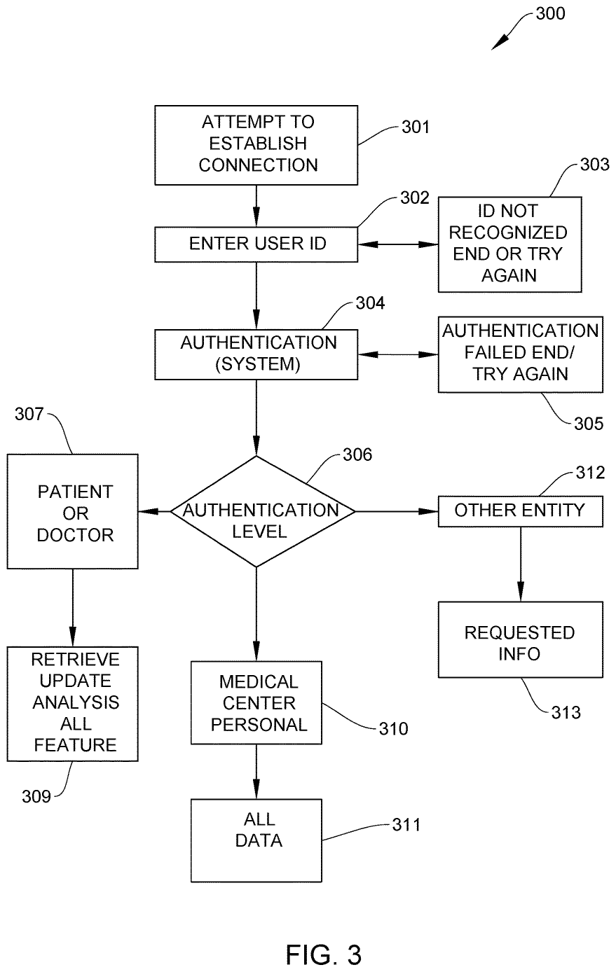

FIG. 3 is a flowchart that depicts how different types of users may interact with the analysis processing centre (102) or main AI system. In (301), the user may attempt to establish a connection through a computer, mobile phone, laptop, tablet, or other device; each user has a user identification, which he enters (302). If the user ID is not recognised, the system may terminate the connection or allow the user to try again (303).

If the user ID is recognised (302), then the user may be required to authenticate himself to the system (304), for example, with a password, a biometric identification, or other authentication steps. If the authentication fails, the system may terminate the connection or allow the user to try again (305).

If the authentication succeeds, the authorization level of the user (306) may determine what functions of the system the user may access. A user identified as a patient or doctor (307) may retrieve, update, analyses, and otherwise access all patient-related data and features for a specific patient. The administrator of the analysis processing centre may have access to components of said center (102) or main AI system in step (311). Other entities (312) may be able to browse or search for requested information without making changes to the data (313).

The AI component of this system is designed to leverage all accessible scientific data, and it embodies the future platforms TreatmentPlan.ai, GoldStandard.ai, and StateOfTheArt.ai. These platforms will represent the gold standard and state-of-the-art medical knowledge derived from a vast array of scientific data. This cutting-edge information will be summarized and made accessible through these portals. Then, this summarized knowledge will be used as a benchmark against which individual patient diagnoses and treatment plans are compared, enabling the AI to identify areas needing further attention or adjustment.

The above descriptions and illustrations should not be taken as exhaustive or as limiting the scope of the present invention to the precise forms disclosed. Those of ordinary skill in the art will appreciate that the invention may be manifested in other specific forms, adaptations, and modifications without departing from its essential characteristics. Thus, the disclosed embodiments are illustrative, not restrictive, and the invention is not confined to the exact structures and operations shown and described.

1-32. (canceled)

33. An AI-based system for patient diagnosis and treatment recommendation, comprising: a main AI engine configured to receive, aggregate, and analyze medical information and personal medical data; a plurality of AI-enabled data sources providing electronic health records, laboratory data, diagnostic imaging data, pathology data, molecular biology data, genomic data, medication-related data, and anamnesis results; and one or more servers configured to execute an AI model that processes the aggregated data to generate diagnostic suggestions and treatment recommendations.

34. The system of claim 33, wherein the main AI engine provides real-time, bidirectional communication with authorized healthcare professionals.

35. The system of claim 33, wherein the plurality of AI-enabled data sources includes electronic health record systems providing medical history, diagnoses, treatments, and medication information.

36. The system of claim 33, wherein the plurality of AI-enabled data sources includes diagnostic imaging systems providing X-rays, MRIs, CT scans, and other radiological images.

37. The system of claim 33, wherein the plurality of AI-enabled data sources includes pathology analysis systems configured to provide interpreted pathological data.

38. The system of claim 33, wherein the plurality of AI-enabled data sources includes molecular biology systems providing proteomics and metabolomics information.

39. The system of claim 33, wherein the plurality of AI-enabled data sources includes genomic analysis systems configured to provide genomic sequencing or variant analysis.

40. The system of claim 33, wherein the plurality of AI-enabled data sources includes systems providing medication-related information including dosage, effectiveness, side effects, and drug-drug interactions.

41. The system of claim 33, wherein the plurality of AI-enabled data sources includes an anamnesis AI module configured to interact with a patient through dynamic questioning to obtain structured patient inputs.

42. The system of claim 33, wherein the main AI engine integrates data from the plurality of AI-enabled data sources to compute risk scores, confidence levels, and ranked diagnostic and treatment recommendations.

43. The system of claim 33, wherein the plurality of AI-enabled systems comprises PatientConnect.ai, GenomicAnalysis.ai, LabResults.ai, PatientRecords.ai, DiagnosticImaging.ai, DiagnosticPathology.ai, MolecularBiology.ai, Pharmacotherapy.ai, and Anamnesis.ai.

44. A method for AI-enabled patient diagnosis and treatment recommendation, comprising: receiving patient-related data from a plurality of AI-enabled data sources; transmitting the data to a main AI engine; processing the data using an AI model; and generating diagnostic suggestions and treatment recommendations.

45. The method of claim 44, further comprising obtaining electronic health records from an independent AI-enabled EHR system.

46. The method of claim 44, further comprising receiving diagnostic imaging data including radiological images from an AI-enabled imaging system.

47. The method of claim 44, further comprising receiving laboratory analysis data from an AI-enabled laboratory data system.

48. The method of claim 44, further comprising receiving genomic analysis data from a genomic analysis AI system.

49. The method of claim 44, further comprising receiving molecular biology data, including proteomics and metabolomics information, from an AI-enabled molecular biology system.

50. The method of claim 44, further comprising receiving medication information including effectiveness, side effects, and drug-drug interactions from an AI-enabled pharmacotherapy system.

51. The method of claim 44, further comprising performing automated patient anamnesis using an anamnesis AI system and transmitting the results to the main AI engine.

52. The method of claim 44, wherein the main AI engine integrates the data from the plurality of AI-enabled systems to produce a unified diagnostic and therapeutic output for healthcare professionals.
